(19) United States
(12) Patent Application Publication
(10) Pub. No.: US 2011/0127700 A1
(43) Pub. Date: Jun. 2, 2011

(54) METHOD FOR PRODUCING A CERAMIC COMPONENT

(75) Inventor: Helmut D. Link, Hamburg (DE)

(73) Assignee: DERU GMBH, Norderstedt (DE)

(21) Appl. No.: 13/057,913

(22) PCT Filed: Aug. 7, 2009
(86) PCT No.: PCT/EP09/05746

§ 371 Date: Feb. 7, 2011

(30) Foreign Application Priority Data
Aug. 7, 2008 (EP) PCT/EP2008/006524

(51) Int. Cl. C04B 41/53; A61F 2/44

(52) U.S. Cl. 264/662; 623/17.16

(57) ABSTRACT

The invention relates to a method for producing a ceramic component. With the method, a ceramic base material in powdered form and a mold having the shape of the ceramic component are provided. The ceramic base material is introduced into the mold. The ceramic component is presintered at a temperature between 880° C. and 980° C. and is then removed from the mold. The surface of the ceramic component is treated with a blasting material and the ceramic component is sintered at a temperature that is higher than the presintering temperature. With the method according to the invention, ceramic components having higher surface roughness can be produced. Due to the higher surface roughness it is easier to apply a firmly adhering coating to the ceramic component.

The invention relates to a method for producing a ceramic component. In the method, a ceramic starting material in powder form is made available and is introduced into a mold that predefines the shape of the ceramic component. The ceramic component is presintered, removed from the mold, and then sintered at a temperature that is higher than the temperature of the presintering.

Such a method for producing a ceramic component is known from EP 0 421 085, for example. It has been found that ceramic components produced in this way have a low surface roughness and that it is therefore difficult to apply a coating firmly to the ceramic component.

The object of the invention is to provide a method for producing a ceramic component, in which method a ceramic component produced according to the invention offers a better starting basis for the application of a coating. Proceeding from the aforementioned prior art, the object is achieved by the features of the independent claims. Advantageous embodiments are set forth in the dependent claims. According to the invention, a temperature of between 880° C. and 980° C. is chosen for the presintering step. After the presintering, and before the sintering, the surface of the ceramic component is treated with a blasting material.

In order to produce ceramic components, a ceramic starting material in powder form is first introduced into a mold that predefines the shape of the ceramic component. Since the volume of the ceramic component decreases as a result of subsequent processing steps, the mold is larger than the finished ceramic component. A stable inner structure of the ceramic component is achieved by subsequent sintering. The sintering results in firm connections between the particles of the powder.

The sintering is performed in several steps. In a first step, the ceramic component is presintered at a lower temperature, such that only narrow bridges are formed between the grains of the powder. For the method according to the invention, the stability of the narrow bridges should be precisely such that the treatment of the surface with a blasting material leads to an increased surface roughness. If the stability of the bridges is too low, the ceramic component can burst into several parts, and, if the stability of the bridges is too high, the treatment with the blasting material is not sufficient to change the surface structure of the ceramic component. Tests have shown that the desired change of the surface structure by the blasting material occurs when a temperature of between 880° C. and 980° C. is chosen for the presintering. The inner structure of the ceramic component is then precisely such that fragments of the desired size can be broken out from the component by the blasting material.

The strength of the connection between the particles of the powder material generally increases when the temperature of the presintering is raised. Good results were obtained by the method according to the invention when the temperature of the presintering was higher than 900° C. or lower than 950° C.

In order to be able to coat the ceramic component with a titanium alloy, the surface roughness Rₐ should be greater than 2.5 μm (WO 2009/036845). The roughness values in the context of the invention relate to the mean roughness Rₐ in accordance with DIN EN ISO 4288 and 3274 and to the finished ceramic component after the sintering. The surface roughness achieved by the method according to the invention depends on the parameters chosen in the treatment with the blasting material, for example the particle size of the blasting material and the speed with which the particles strike the ceramic component. In the method according to the invention, these parameters are preferably adapted such that the surface roughness Rₐ of the finished ceramic component is greater than 2.5 μm. To ensure that the coating can be applied to cover the surface in one operating step, the surface roughness Rₐ of the finished ceramic component should be no greater than 7 μm.

In the context of the invention, the entire surface of the ceramic component can be treated with the blasting material. This is expedient if the entire surface of the ceramic component is to be provided with a coating. However, it is often the case that only a part of the surface is to be coated, while another part of the surface is to remain free of coatings. This applies, for example, to endoprosthesis components whose surface is intended in part to form a connection with bone substance, while another part is intended to serve as a slide surface for interaction with another prosthesis component. A high degree of surface roughness is not desired for the slide surface. In the method according to the invention, provision can therefore be made that only a part of the surface of the ceramic component is treated with the blasting material, while another part of the surface remains excluded from the treatment with the blasting material. When treatment of the surface of the ceramic component is referred to in the context of the invention, this can signify the entire surface or a part of the surface.

The particle size of the blasting material is preferably of the same order as the particle size of the ceramic starting material. The blasting material can then act particularly effectively on the ceramic component. However, when such a blasting material is used, there is a danger of particles of the blasting material adhering in the gaps that occur in the ceramic material as a result of the fragments that are broken out. The particles then represent impurities in the ceramic material. Impurities of this kind can be prevented by using as blasting material a powder that corresponds to the powder of the ceramic starting material. If particles of this material adhere in the ceramic component, they do not alter the homogeneous composition of the ceramic material.

The coating to be applied to the surface of the ceramic component can be composed of a titanium alloy or of pure titanium. A possible method for applying the coating is plasma spraying. Plasma spraying is a method in which a gas is guided through an electric arc and thereby ionized. The coating material is introduced in powder form into the ionized gas and is transported by the stream of gas onto the workpiece that is to be coated.

Zirconium oxide, aluminum oxide, and mixtures of the two, have proven suitable as ceramic materials for the method according to the invention.

The invention is described in more detail below with reference to the attached drawing and on the basis of an advantageous illustrative embodiment. In the drawing:

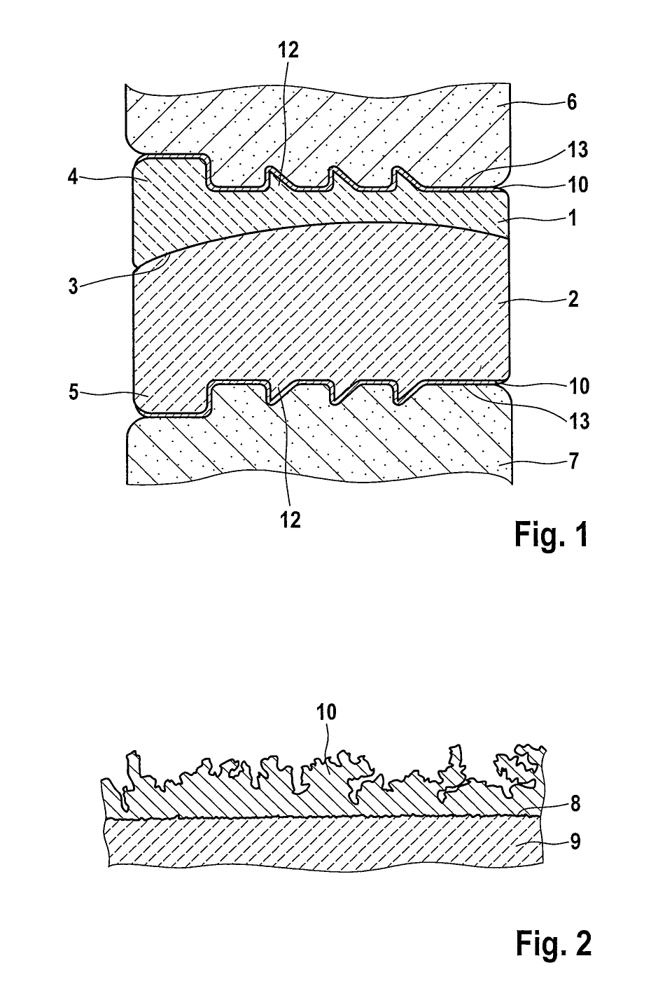

FIG. 1 shows an intervertebral disk prosthesis in cross section;

FIG. 2 shows an enlarged detail of the intervertebral disk prosthesis according to FIG. 1;

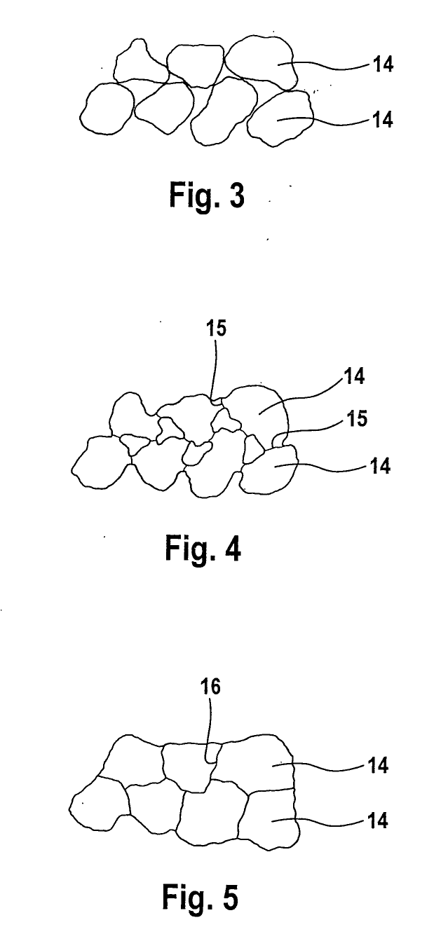

FIG. 3 shows a schematic view of the inner structure of the ceramic material in the starting state;

FIG. 4 shows the view from FIG. 3 after the presintering; and

FIG. 5 shows the view from FIG. 3 after the sintering.

In FIG. 1, an endoprosthesis designed as an intervertebral disk prosthesis is inserted into an intervertebral space between two vertebral bodies 6, 7. The intervertebral disk prosthesis comprises a first contact plate 1 and a second contact plate 2. The first contact plate 1 is an endoprosthesis component designed for connection to a first vertebral body 6, and the second contact plate 2 is an endoprosthesis component designed for connection to a second vertebral body 7.

The first contact plate 1 and the second contact plate 2 bear on each other via matching slide surfaces 3. The slide surfaces 3 form a hinge for movements between the upper contact plate 1 and the lower contact plate 2.

Projections 12 are formed in areas 13 of the surfaces of the contact plates 1, 2 at which the contact plates 1, 2 bear on the bone substance of the vertebral bodies 6, 7. The projections 12 have a shallower flank in the direction in which the contact plates 1, 2 are pushed into the intervertebral space, and a steeper flank in the opposite direction. The shallower flank makes it easier to push the contact plates 1, 2 into the intervertebral space. The steeper flank provides the contact plates 1, 2 with a hold when the projections 12 have penetrated into the bone substance of the vertebral bodies 6, 7. The steeper flanks prevent the contact plates 1, 2 from being pulled back out again from the intervertebral space in the opposite direction.

Flanges 4, 5 of the contact plates 1, 2 are intended to bear on the ventral aspect of the vertebral bodies 6, 7. The flanges 4, 5 have the effect that the contact plates 1, 2 cannot be pushed into the intervertebral space in the dorsal direction any further than the desired position.

The contact plate 1 and the contact plate 2 are formed from a ceramic material 9. The slide surfaces 3 on the contact plates 1, 2 are formed on a surface of the ceramic material 9. The areas 13 at which a connection to the bone substance is established are coated with a titanium alloy coating 10.

FIG. 2 shows an enlarged detail of a contact plate 1, 2, in which the interface 8 between the ceramic material 9 and the coating 10 is shown. The ceramic material 9 has a roughness Rₐ of at least 2.5 μm at the interface 8 to the coating 10. This roughness at the interface 8 is sufficient to establish a stable connection to the coating 10, but it is not sufficient for a stable connection to bone substance. The coating 10 has a greater roughness and greater porosity than the ceramic material 9 and thus forms a stable connection to the bone substance.

In order to produce a ceramic component in the form of an endoprosthesis component in which a part of the surface has the desired roughness, a starting material present in powder form is first introduced into a mold whose shape corresponds to the ceramic component that is to be produced. Since the ceramic component loses volume as a result of the subsequent method steps, the mold has greater dimensions than the finished ceramic component. The powder is mechanically compacted in the mold, for example by shaking and pressure, and the particles 14 of the powder then lie next to one another, as is shown schematically in FIG. 3. As a result of the presintering at a temperature of 920° C., bridges (shown in FIG. 4) form between the particles 14. The inner structure of the material is now so stable that the ceramic component can be removed from the mold.

With the bridges 15, the ceramic material has a greater stability of its structure than one would choose for mechanical working by drilling or milling. The stability of the structure is specifically such that, by blasting with a powder that corresponds to the starting material, fragments can be detached from the presintered ceramic material. The fragments are so large that, after the sintering, the finished ceramic component has the desired surface roughness.

When the desired surface structure is achieved and the mechanical working is completed, the ceramic component is sintered at a temperature that is higher than the temperature of the presintering. Planar connections 16 form at the interfaces of the particles which were previously connected only via the bridges 15. Since the cavities disappear from the interior, the volume of the ceramic component further decreases. On the part of the surface that was treated with the blasting material, the surface now has a roughness Rₐ of about 4 μm. A coating composed of a titanium alloy applied to this portion of the surface adheres firmly to the surface.

1. A method for producing a ceramic component, said method comprising
introducing a ceramic starting material in powder form into a mold that predefines the shape of the ceramic component;
presintering the ceramic component;
removing the ceramic component from the mold;
treating the surface of the ceramic component with a blasting material;
sintering the ceramic component at a temperature that is higher than the temperature of the presintering.

2. The method of claim 1, wherein the temperature of the presintering is higher than about 900° C.

3. The method of claim 1, wherein the temperature of the presintering is lower than about 950° C.

4. The method of claim 1, wherein the roughness Rₐ of the ceramic component on a portion of the surface is greater than about 2.5 μm.

5. The method of claim 1, wherein the blasting material particle size corresponds to the particle size of the ceramic starting material.

6. The method of claim 1, wherein the ceramic starting material is used as the blasting material.

7. The method of claim 1, wherein, a coating is applied to the surface of the ceramic component.

8. The method of claim 1, wherein the entire surface of the ceramic component is treated with the blasting material.

9. The method of claim 1, wherein less than the entire surface of the ceramic component is treated with the blasting material.

10. The method of claim 1, wherein the presintering is at a temperature of between about 880° C. and about 980° C.

11. The method of claim 7, wherein the coating comprises titanium or titanium allow.

12. The method of claim 11, wherein the titanium or titanium alloy coating is applied to the surface of the ceramic component utilizing plasma spraying.

13. The method of claim 7, wherein the coating has a greater roughness and greater porosity than the ceramic component.

14. The method of claim 1, wherein the ceramic starting material in powder form is chosen from at least one of the following:
zirconium oxide, aluminum oxide, and a mixture of zirconium oxide and aluminum oxide.

15. The method of claim 1, wherein the ceramic component is an endoprosthesis component.

16. The method of claim 1, wherein the roughness Rₐ of the ceramic component on a portion of the surface is no greater than about 7.0 μm.

17. A prosthesis, comprising:
a ceramic component formed from a ceramic starting material in powder form selected from the group comprising zirconium oxide, aluminum oxide, and a mixture of zirconium oxide and aluminum oxide, wherein the ceramic starting material is inserted into a mold that predefines the shape of the ceramic component and presintered to form the ceramic component, and wherein the ceramic component is removed from the mold, the surface of the ceramic component is treated with a blasting material, and the ceramic component is sintered at a temperature that is higher than the temperature of the presintering; and
a coating comprising titanium or titanium alloy, wherein the coating is applied to the surface of the ceramic component and wherein the coating has a greater roughness and greater porosity than the ceramic component.

18. The prosthesis of claim 17, wherein the roughness Rₐ of the ceramic component on a portion of the surface is between about 2.5 μm and about 7.0 μm.

19. The prosthesis of claim 17, wherein the blasting material particle size corresponds to the particle size of the ceramic starting material.

20. The prosthesis of claim 17, wherein the presintering is at a temperature of between about 880° C. and about 980° C.

21. The prosthesis of claim 17, wherein the coating has a greater roughness and greater porosity than the ceramic component.

22. The prosthesis of claim 17, wherein the titanium or titanium alloy coating is applied to the surface of the ceramic component utilizing plasma spraying.
